(19) United States
(12) Patent Application Publication
(10) Pub. No.: US 2024/0009094 A1
(43) Pub. Date: Jan. 11, 2024

(54) ANTICAKING COMPOSITION AND METHOD FOR IMPROVING FLOWING PROPERTIES OF HYGROSCOPIC TIN SALTS

(71) Applicant: HONEYWELL INTERNATIONAL INC., Charlotte, NC (US)

(72) Inventors: Jurgen Rudolph, Peine (DE); Heike Dähndel, Seelze (DE)

(73) Assignee: HONEYWELL INTERNATIONAL INC., Charlotte, NC (US)

(21) Appl. No.: 18/311,925

(22) Filed: May 4, 2023

Related U.S. Application Data
(60) Provisional application No. 63/368,067, filed on Jul. 11, 2022.

(51) Int. Cl. A61K 8/20; A61K 8/26; A61K 8/19; A61K 8/24; A61K 8/21; A61K 8/27; A61Q 11/00

(52) U.S. Cl. CPC A61K 8/20; A61K 8/26; A61K 8/19; A61K 8/24; A61K 8/21; A61K 8/27; A61Q 11/00

(57) ABSTRACT

The present disclosure generally relates to a particulate composition that includes a particulate source of stannous, alumina, and a drying aid. The disclosure further relates to a method for improving the flowability of particulate stannous by admixing the alumina and the drying aid with it thereby giving improved flow properties to the stannous chloride.

This application claims the benefit of U.S. Provisional application No. 63/368,067, filed 11 Jul. 2022, the entire contents of which are hereby incorporated by reference.

### TECHNICAL FIELD

The present disclosure relates to a particulate composition that includes a particulate source of stannous, alumina, and a drying aid. The technology further relates to a method for improving the flowability of particulate stannous by admixing the alumina and the drying aid with it thereby giving improved flow properties to the stannous chloride.

### BACKGROUND

Tin (II) (stannous) ions, provided in oral compositions by stannous chloride and/or other stannous salts, have long been valued for the multiple benefits that they can afford, including antimicrobial effects, control of breath malodor, control of dental plaque growth and metabolism, reduced gingivitis, decreased progression to periodontal disease, reductions in dentinal hypersensitivity, and reduced coronal and root dental caries and erosion.

The use of a stannous salt as a raw material can pose problems. For Example, stannous (II) chloride, both dihydrate and anhydrous forms, are hygroscopic materials and thus attract water vapor from the air through both absorption and adsorption. This makes the powdered compound sticky. Particles can bind together, whereas agglomerates can form during transit and storage and thus flowability during processing is difficult. Hygroscopic materials also have a tendency over time to become damp and soft when exposed to air that contains a lot of moisture. Therefore, the moisture level held by these hydroscopic salts is usually proportional to the level of humidity.

It has been a problem in the art to ship, store and handle stannous (II) ion salts due to the handling problems discussed above. Accomplishing flow of stannous ion salts from storage bins has proven to be difficult. Ideal flow design would be a simple storage bin with wall angles steep enough to promote mass flow. However, stannous chloride cakes so readily that simple mass flow design does not work.

Because of these issues, drying aids are used. Common drying agents are generally anhydrous inorganic salts that acquire waters of hydration when exposed to moist air or a wet solution. For the most common drying agents such as sodium sulfate or magnesium sulfate, the crystals form larger clumps when they absorb water. In organic laboratory technique organic solvents being dried by using anhydrous salts. Other drying aids include CaCl₂, CaO, zeolites, and silica gel.

Other ways of dealing with issues related to the hydroscopic nature of stannous salts is the use of an anti-caking agent, which can improve the flow, decrease the compaction and therefore decrease restricted flow during processing. Anti-caking agents function either by adsorbing excess moisture or by coating particles to make them less prone to water adsorption. Other compounds are known to experience similar problems to stannous chloride, for example, potassium nitrate.

Other objectives include finding a composition that is compatible with a desiccant or drying aid for the hygroscopic fluorine salts used in dental care formulations. These desiccants must be non-toxic, highly pure and available in food or pharmaceutical grade. In addition, the desiccants need to have good drying and/or flowability for stannous salts.

### BRIEF SUMMARY

Provided is a composition that includes a particulate source of a stannous salt; an alumina or combination of alumina's and a desiccant or drying agent. The term desiccants and drying aids are used interchangeably throughout the application. The particulate stannous salt can be chosen from stannous chloride, stannous fluoride and/or stannous pyrophosphate. The alumina can either be chosen from a spray dried, a rotary calcinated, a fumed, a pearled alumina or combinations thereof. The drying agent can be any drying aid known in the art.

Also provided, is a method of improving the flowability of particulate stannous salts by adding to a particulate stannous source, such as, stannous chloride, stannous fluoride and/or stannous pyrophosphate, an alumina and a drying aid.

Furthermore, provided is a composition that can be used in oral care formulations, such as toothpaste.

This summary is provided to introduce a selection of concepts in a simplified form that are further described below in the detailed description. This summary is not intended to identify key features or essential features of the claimed subject matter, nor is it intended to be used as an aid in determining the scope of the claimed subject matter.

### DETAILED DESCRIPTION

The following detailed description is merely exemplary in nature and is not intended to limit the invention or the application and uses of the invention. Unless specifically stated or obvious from context, as used herein, the term “about” is understood as within a range of normal tolerance in the art, for example within 2 standard deviations of the mean. “About” can be understood as within 10%, 5%, 1%, 0.5%, 0.1%, 0.05%, or 0.01% of the stated value. “About” can alternatively be understood as implying the exact value stated. Unless otherwise clear from the context, all numerical values provided herein are modified by the term “about.”

In some aspect of the current composition, the composition includes a particulate source of a stannous salt, an alumina, and a drying aid. The stannous salt can be chosen from stannous chloride, stannous fluoride and stannous pyrophosphate. The stannous salt can be from one or more of the stannous salts or a single salt, such as stannous chloride.

The anhydrous grade can be supplied in various forms: powder, flake and pellets. Stannous chloride dihydrate is commercially available from various suppliers. Physical characteristics include a colorless crystalline material with a slight characteristic odor, poor flow characteristics and a relatively short shelf life. However, the disadvantages of using stannous chloride, either in the dihydrate or anhydrous form, are the manufacturing constraints, both in shipping and handling. Both the dihydrate and the different anhydrous forms, to a greater or lesser extent, are hygroscopic, making flowability during processing difficult and giving a poor activity over the shelf life of the material. Stannous chloride is also an aggressive reducing agent to some metals. This property can lead to the formation of undesirable compounds because the material cakes and sits on dead spots in storage bins.

In some aspects of the composition, the stannous chloride can be chosen from stannous chloride dihydrate, stannous chloride anhydrous, and combinations thereof.

In some aspects of the composition, the alumina can have an average particle size of from about 0.4 microns (μm) to about 100 μm, or from about 1 μm to about 90 μm, or from about 2 μm to about 80 μm, or from about 2 μm to about 30 μm as measure by laser diffraction particle size analyzer using a Horiba LA-960. An example, VP AEROPERL® Alu 100/30, from Evonik, which is a hydrophilic fumed alumina granulate that is a crystalline material of alumina and anatase having a white fluffy appearance a highly pure powder or ULTRA ° HPP, from Alpha HPA, which is rotary calcinated high pure gamma alumina with spherically shaped particles.

Although not wishing to be bound by theory, it is believed that the fineness of the metal oxides are able to encase the particles of the stannous salts. The metal oxides, i.e. alumina, act as spacers or carriers between the individual stannous particles, which keeps them separated from each other and ultimately reduce adhesive forces.

Because the alumina has a positive charge, it neutralizes the negative charge of polymer powders or negatively charged particles. This renders the powder to become electrically neutral and therefore resists anticaking and sticking to walls, pipes and each other.

In some aspects of the composition alumina is mixed with the stannous salt within the range of about 0.5-10%, and can be about 0.5-5% by weight alumina admixed.

In some aspects of the composition, the ratio of particulate stannous salt to alumina can be from about 9:1 to 199:1, and can be about 19:1 to 199:1.

In yet other aspects of the composition, the ratio of drying aid to alumina can be from about 10:1 to 2:1, or from about 3:1 to 5:1.

In some aspects of the composition, the drying aid can be chosen from calcium sulfate, magnesium sulfate, zinc stearate, and combinations thereof.

In yet other aspects of the composition, the composition further includes one or more oral care agents chosen from a source of fluoride ions, dental abrasives, flavors, humectants, chelants and combinations thereof.

In some aspects, the current technology includes a method of improving the flowability of particulate stannous salts. The method includes adding an alumina and a drying aid to the particulate source of a stannous salt.

In some aspects of the method, provided is a stannous salt chosen from stannous chloride, stannous fluoride and/or stannous pyrophosphate. The stannous salt can be stannous chloride, such as stannous chloride dihydrate, stannous chloride anhydrous, and combinations thereof.

In some aspects of the method, the alumina added to the stannous salt can be chosen from a spray dried, a rotary calcinated, a fumed, a pearled alumina or combinations thereof.

In some aspects of the method, the particulate stannous salt provided The alumina can have an average particle size of from about 0.4 microns (μm) to about 100 μm, or from about 1 μm to about 90 μm, or from about 2 μm to about 80 μm, or from about 2 μm to about 30 μm.

In some aspects of the method, the ratio of particulate stannous salt to alumina can be from about 9:1 to 199:1, and can be about 19:1 to 199:1 and the ratio of drying aid to alumina can be from about 10:1 to 2:1, or from about 3:1 to 5:1.

In yet other aspects of the method, the drying aid added to the particulate stannous salt, can be chosen from calcium sulfate, magnesium sulfate, zinc stearate, and combinations thereof.

In still other aspects of the method, one or more oral care agents can be added to the composition, for example, a source of fluoride ions, dental abrasives, flavors, humectants, chelants, and combinations thereof.

In other aspects, the composition can be used in oral care formulations, for example, toothpaste.

### EXAMPLES

The following examples were accomplished using known drying aids with stannous fluoride (SnF₂) and stannous chloride (SnCl₂) in a humid atmosphere and measuring the moisture uptake. In the current study, the water absorption of SnF₂ and SnCl₂ in the presence of different desiccants or drying aids was observed.

### Example 1—Water Uptake Test

Samples were prepared in which 3.5 gram (g) of stannous chloride (SnCl₂) was mixed with 0.15 g of a desiccant (Ca₃(PO₄)₂ or CaSO₄) and a sample in which 0.15 g of a flowing aid (flumed alumina) was added to the stannous chloride and desiccant as indicated below in Table 1. The salt mixtures were dried in an oven for a specified time 24 h and temperature 120° C. and then placed in a desiccator to cool to room temperature and immediately weighed. The salt mixtures were then stored at constant humidity of 93% at 23° C. for 6 days or coming to equilibrium and the weight gain recorded over time. Water absorption is expressed as increase in weight present. i.e. % water absorption=[(wet weight−dry weight)/dry weight]×100. Results can be seen in Table 1 below.

TABLE 1
Water Uptake

| | | | | SnCl₂ + |
|---|---|---|---|---|
| | | | | Al₂O₃ + |
| | SnCl₂ | Ca₃(PO₄)₂ | SnCl₂ + CaSO₄ | CaSO₄ |
| Storage Time | (% Water | (% Water | (% Water | (% Water |
| (days) | Uptake) | Uptake) | Uptake) | Uptake) |
| 0 | 0.0 | 0.0 | 0.0 | 0.0 |
| 1 | 4.23% | 5.0 | 3.6 | 3.09% |
| 2 | 8.54% | 8.8 | 5.0 | 6.82% |
| 3 | 10.67% | 12.4 | 5.9 | 9.40% |
| 6 | 16.85% | 20.8 | 8.8 | 13.10% |

The results indicate that a composition that includes a flowing aid can reduce the uptake of water of stannous chloride over 2-component systems in the first 24 hours and reduces water uptake significantly over the composition when the 2-component system only includes Ca₃ (PO₄)₂ as the desiccant.

### Example 2—Water Uptake Test

As in Example 1, the water absorption uptake in a constant humidity atmosphere was measured over a six day period. The stannous chloride was admixed with various drying aids and aluminum oxides as indicated in Table 2 below.

TABLE 2
Particulate Stannous Chloride Water Uptake

| Sample | Additive | Weight Gain (%) | Physical Property |
|---|---|---|---|
| | no drying aid | 16.9 | wet |
| 1 | Zeodent ® 119, | 8.1 | fine, dry |
| | flowing aid | | |
| 2 | Ca₂SO₄, drying aid | 8.8 | fine, dry |
| 3 | Aerosil ® 200 SiO₂ | 9.4 | fine, dry |
| 4 | Aeroxid ®, Al₂O₃ | 10.2 | wet |
| 5 | Na₂SO₄, drying aid | 12.8 | wet |
| 6 | MgSO₄, drying aid | 13.5 | wet |
| 7 | Zinc oxide, drying | 18.7 | wet |
| | aid | | |
| 8 | Tricalcium | 20.8 | wet |
| | phosphate, drying aid | | |
| 9 | Aeroxid ®, Al₂O₃ + | 13.9 | dry |
| | drying aid | | |
| 10 | Aeroperl ®, Al₂O₃ + | 13.1 | dry |
| | drying aid | | |

Results indicate that the drying aids Zeodent® (silica), calcium sulfate, Aeroperl® Aeroxid® (aluminum oxides) in combination with a drying aid, and Aerosil® (silica) are able to reduce water absorption compared to pure stannous chloride. Although, the weight gained by the stannous chloride was better in some instances and worse in others when alumina was combined with a drying aid, the physical properties were stable and significantly improved when blending the stannous fluoride with alumina, which kept the blend dry.

### Example 3—Water Uptake Test

The same procedure was followed as in Examples 1 and 2 above, except stannous fluoride replace the stannous chloride. The drying aids listed in Table 3, were admixed with the stannous fluoride and the water uptake, as determined by the weight gain of the sample after six days in the constant humidity atmosphere.

TABLE 3
Particulate Stannous Fluoride Water Uptake

| Sample | Additive | Weight Gain (%) | Physical Property |
|---|---|---|---|
| | no add | 3.8 | lumpy |
| 1 | Zeodent ® 119, | 1.2 | fine, dry |
| | flowing aid | | |
| 2 | Ca₃(PO₄)₂, drying | 1.6 | fine, dry |
| | aid | | |
| 3 | Aerosil ® 200 SiO₂, | 2.7 | fine, dry |
| | flowing aid | | |
| 4 | MgSO₄, drying aid | 2.9 | lumpy |
| 5 | Na₂SO₄, drying aid | 3.5 | lumpy |
| 6 | ZnO, drying aid | 3.7 | lumpy |
| 7 | Aeroxid ® Al₂O₃, | 4.3 | fine, dry |
| | flowing aid | | |
| 8 | CaSO₄, drying aid | 6.2 | lumpy |
| 9 | Al₂O₃ + CaSO₄ | 2.7 | Fine dry |

Results indicated that the flowing aids Zeodent® and tricalcium phosphate were the only drying aids that did not have perceptible weight gain over the stannous fluoride alone. However, when the stannous fluoride was blended with alumina or alumina and calcium sulfate, the composition was able to maintain its physical property of being fine, dry and powdery.

### Example 4—Flowability Test

In the following test was done using a Copely Flowability Tester Model BEP according to specifications detailed in the European Pharmacopoeia 2.9.16-1. In this test, 100 g of stannous chloride was blended with 1.5-5.0% desiccant and 0.5% to 5.0% flowing aid. This formulation was compared with a sample of stannous chloride blended with 1% Zeodent® 119 having a particle size of 6-15 microns (μm). The stannous chloride was blended with a drying aid and flowing aid in a tumble mixer for 10 minutes. Then 100 g of the blend was transferred to the funnel, the shutter is removed and the flow through time recorded with a stopwatch. Results can be found in Table 4.

TABLE 4
Flow Test

| | % | Flowing | % | | Flow | Physical |
|---|---|---|---|---|---|---|
| Additive | Desiccant | aid | Alumina | | Times | Characteristics |
| CaCO₃ | 5.0 | Aeroperl | 1.5 | Blend | 10 | Clumpy |
| Ca-D- | 5.0 | Aeroperl | 1.5 | Blend | 10 | Clumpy |
| Gluconate | | | | | | |
| MgO | 5.0 | Aeroperl | 1.5 | Blend | 10 | Clumpy |
| Na₂CO₃ | 5.0 | Aeroperl | 1.5 | Blend | 10 | Clumpy |
| Na₂SO₄ | 5.0 | Aeroperl | 1.5 | Blend | 10 | Clumpy |
| Mg₃(PO₄)₂ | 5.0 | Aeroperl | 1.5 | Blend | 10 | No Flow |
| Zinc | 5.0 | Aeroperl | 1.5 | Blend | 10 | No Flow |
| citrate | | | | | | |
| Zeolite | 5.0 | | | Single | 10 | Clumpy |
| ZSM X13 | | | | Use | | |
| CaSO4 | 5.0 | | | Single | 10 | No Flow |
| | | | | Use | | |
| Ca₃(PO₄)₂ | 5.0 | Aeroperl | 5.0 | Blend | 6 | Flows |
| Zinc | 5.0 | Aeroperl | 1.5 | Blend | 5 | Fast Flow |
| stearate | | | | | | |
| Aeroxid ® | | | 5.0 | Single | 5 | Fast Flow |
| | | | | Use | | |
| Zeodent ® | 2.5 | | | Single | 5 | Fast Flow |
| 119 | | | | Use | | |
| Aerosil ® | 2.5 | | | Single | 5 | Fast Flow |
| 200 | | | | Use | | |
| CaSO₄ | 5.0 | Aeroperl | 1.5 | Blend | 4 | Fast Flow |
| MgSO₄ | 5.0 | Aeroperl | 2.5 | Blend | 4 | Fast Flow |
| Aeroperl ® | | | 5.0 | Single | 4 | Fast Flow |
| 100/30 | | | | Use | | |

* drying aid level was 5.0 wt. % based on total composition.

Results indicated that Zeodent®, Aerosil® and Aeroxid® stimulated the flowability of SnCl₂ while using a blend of Aeroxid® 2.5% in combination with a drying aid, improves the flow ability of SnCl₂.

### Example 5—Addition of Aluminum Oxide

The following test was done using samples of 100 g stannous chloride as the salt, admixed with CaSO₄ and Zn-stearate. Aeroperl® was added to the formulations and compared with a formulation without it. Flow time and physical property of the formulations were determined. Results are shown in Table 5.

Results indicate that a concentration of 5% calcium sulphate alone does not stimulate the flow properties, whereas Aeroperl® does from a concentration of at least 5%. If, on the other hand, 2.5% of a drying aid is first mixed with SnCl₂ and then Aeroperl is gradually added, then 2.1% Aeroperl is already sufficient to stimulate the powder mixture to flow. If the tin chloride is first mixed with the drying aids zinc stearate, then the mixture already begins to flow with 1.5% Aeroperl®.

### Example 6—Water Absorption in Constant Humidity

In this example, the procedure in Example 1 was followed, i.e. a mixture of a drying aids (as indicated in Table 6) and alumina (Aeroperr), was admixed with stannous chloride. The salt mixtures were stored at a constant humidity of 93% at 23° C. for 6 days and the weight gain recorded over time. Results are shown in Table 6.

TABLE 6
Water Absorption Test

| | | | Tricalcium | Magnesium | Zinc | Natrium | |
|---|---|---|---|---|---|---|---|
| | | CaSO₄/ | sulfate/ | sulfate/ | stearate/ | sulfate/ | |
| Time | | Aeroperl ® | Aeroperl ® | Aeroperl ® | Aeroperl ® | Aeroperl ® | SnCl2 |
| (days) | Aeroperl ® | (%) | (%) | (%) | (%) | (%) | (%) |
| 0 | 0 | 0 | 0 | 0 | 0 | 0 | 0 |
| 1 | 2.89 | 3.18 | 4.64 | 3.65 | 2.58 | 3.66 | 4.2 |
| 2 | 5.58 | 5.71 | 8.39 | 7.06 | 5.57 | 6.62 | 8.5 |
| 3 | 7.40 | 7.11 | 11.51 | 9.53 | 7.12 | 8.50 | 10.7 |
| 6 | 11.50 | 10.46 | 18.95 | 16.57 | 10.16 | 14.58 | 16.9 |

Results indicate that when alumina is used in combination with a drying aid, there is a decrease in water absorption of the stannous chloride. In particular, admixing alumina alone with stannous chloride or admixed with a drying aid such as CaSO₄, Zn-stearate provided advantageous results.

### Example 7—Stannous Ion Stability Test

The following test was done to determine the stannous ion stability in the solid particulate composition of the present formulation. In this study a Raw Material Stability assay was carried out, which compared stannous fluoride mixed with 10% silica (Zeodent® 119) at 23° C. and 60% relative humidity and at 80° C. and 75% relative humidity. Soluble stannous levels were measured by complexometric titration.

In a 250 ml beaker, 36 g SnF₂ and 4 g of each drying aid were added (see Table 7), mixed and stored for 24 hours at 80° C. in a drying oven. After this time, 60 g of H₂O was added, dispersed and the dispersion was stirred for another 12 hours. After the solution had settled, the supernatant solution was removed with a syringe and transferred to the titration template using a 0.25 μm prefilter and determined potentiometrically.

When Stannous salts are stored at high temperatures, e.g., 80° C., it is expected that part of the drying aid will react with the tin, and be bound by it. If the stannous salt e.g., SnCl₂, SnF₂ or Sn₇P₂O₇, is then dissolved in water, the concentration of free tin is now reduced. Part of the tin(II) salt is also oxidized to tin(VI) in air. This portion is not detected by the titration. Ultimately, tin(IV) ions are pathologically ineffective and therefore also lost for the intended application.

Samples of SnF₂ was blended with Zeodent® 119, Aerosil® 200, and tricalcium phosphate and the free stannous ions determined as described above. Results of study can be found in Table 7a and 7b.

TABLE 7a
Stannous Salt Activation After Storage at 80° C.

| Sample | Assay % Sn²⁺ | Physical property |
|---|---|---|
| SnF₂ (Rm Temperature) | 96.63 | Good flow |
| SnF₂(80° C.) | 105.26 | Clumpy, no flow |
| SnF₂/10% Zeodent ® | 56.45 | Good flow |
| SnF₂/10% Aerosil ® | 57.32 | Good flow |
| SnF₂/10% tricalcium | 42.09 | Clumpy, no flow |
| phosphate | | |
| SnF₂/Aeroxid ®/tricalcium | 61.3 | Good flow |
| phosphate | | |
| SnF₂/Aeroperl ®/Tricalcium | 46.20 | Good flow |
| Phosphate | | |

Results indicate that when the stannous salt is admixed with alumina there is a significant decrease in the free stannous ions and particularly with tricalcium phosphate.

TABLE 7b
Stannous Chloride Activation After Storage at 40° C.

| Sample | Assay % Sn²⁺ | Physical property |
|---|---|---|
| SnCl₂ (22° C.) | 97.10 | Clumpy, no flow |
| SnCl₂ (40° C.) | 92.38 | Clumpy, no flow |
| SnCl₂ + 10% Aeroxid ® | 64.71 | Clumpy, no flow |
| SnCl₂ + 10% Aeroperl ® | 85.56 | Good flow |
| SnCl₂ + 5% Aeroperl ® + | 72.59 | Good flow |
| 5% CaSO₄ | | |

### Example 8—Soluble Stannous in Oral Care Formulations

This example indicates the loss of soluble stannous availability in the presence of an anticaking composition.

In 250 ml beakers, solutions of 36 wt. % glycerin, 0.6 wt. % sodium gluconate and 10 wt. % of different anticaking agents were prepared. The total weight of each sample was adjusted with water and a mixture of sodium fluoride and stannous chloride (0.2543 and 0.654 wt/.%, respectively) based on the total weight of the composition, was added to each sample.

50 ml of each of the prepared mixture was placed in a 150 ml beaker, stirred for 24 hours with a magnetic stirrer at 500 rpm and then a 3 ml sample was taken using a disposable syringe with a 0.45 μm syringe filter attached. These samples were analyzed for free tin by potentiometric titration or via inductive coupled plasma. Results can be seen in Table 8a and 8b.

TABLE 8a
Free Ion Concentrations

| | | | Aeroperl ® + |
|---|---|---|---|
| | Zeodent ® | CaSO₄ | CaSO₄ |
| | Free Ion | Free Ion | Free Ion |
| | Concen- | Concen- | Concen- |
| SnCl₂ | tration (%) | tration (%) | tration (%) |
| Drying aid - 1.0% | 87.6% | 82.5% | 100.1% |
| Drying aid - 2.5% | 72.3% | 92.1% | 91.8% |
| Drying aid - 5.0% | 59.6% | 94.1% | 88.3% |
| Drying aid - 7.5% | 47.6% | 96.4% | 82.1% |
| Drying aid - 15.0% | 39.3% | 90.3% | 79.1% |

TABLE 8b
Free Ion Concentrations

| Sample | Flowing Aid | % Sn²⁺ |
|---|---|---|
| SnCl₂ | 10% Aeroperl ® | 28.2% |
| SnCl₂ + CaSO₄ 5% | 5% Aeroperl ® | 88.3% |
| SnCl₂ | 10% Aeroxid ® | 28.0% |
| SnCl₂ + CaSO₄ 5% | 5% Aeroxid ® | 89.3% |

Results indicate that the free tin ion concentration is only reduced by approx. 10% with the addition of up to 5% alumina. In contrast, 10% anti-caking agents lead to a decrease in free tin ions of up to 70%. The drying aid calcium sulphate has rather little or no influence on the stannous ion activity. It should be noted that a 100-fold excess of drying aid was used for this test.

The foregoing tests identified calcium sulfate, zinc stearate, magnesium sulfate, pyrogenic aluminum hydroxide (Aeroxid®) and pyrogenic silicium dioxide (Aerosil®) as the most effective anticaking agents for SnCl₂.

In addition, the combination of the forementioned anticaking agents with aluminium hydroxide (Aeroperl® A100/30) further improves the flowing properties of stannous salts.

Finally, this study unexpectedly reveals that the suppression of the free tin activity with calcium sulfate is lower with that of a silica based drying aid or desiccant. Finally, the combination of calcium sulfate with Aeroperl® not only improves flowing properties, but also lowers the formulation cost of the stannous chloride blend and improves the workability in the production.

While at least one exemplary embodiment has been presented in the foregoing detailed description of the inventive subject matter, it should be appreciated that a vast number of variations exist. It should also be appreciated that the exemplary embodiment or exemplary embodiments are only examples, and are not intended to limit the scope, applicability, or configuration of the inventive subject matter in any way. Rather, the foregoing detailed description will provide those skilled in the art with a convenient road map for implementing an exemplary embodiment of the inventive subject matter. It being understood that various changes may be made in the function and arrangement of elements described in an exemplary embodiment without departing from the scope of the inventive subject matter as set forth in the appended claims.

What is claimed is:

1. A composition comprising:
a) a particulate source of a stannous salt;
b) an alumina; and
c) a drying aid.

2. The composition according to claim 1, wherein the stannous salt is chosen from stannous chloride, stannous fluoride and/or stannous pyrophosphate.

3. The composition according to claim 2, wherein the particulate stannous salt is stannous chloride.

4. The composition according to claim 3, wherein the stannous chloride is chosen from stannous chloride dihydrate, stannous chloride anhydrous, and combinations thereof.

5. The composition according to claim 1, wherein the alumina is chosen from a spray dried, a rotary calcinated, a fumed, a pearled alumina or combinations thereof.

6. The composition according to claim 1, wherein the particulate source has an average particle size of from about 0.4 microns (μm) to about 100 μm.

7. The composition according to claim 1, wherein the ratio of particulate stannous salt to alumina is from about 9:1 to 199:1.

8. The composition according to claim 1, wherein the ratio of drying aid to alumina is from about 10:1 to 2:1.

9. The composition according to claim 1, wherein the drying aid is chosen from calcium sulfate, magnesium sulfate, zinc stearate, and combinations thereof.

10. The composition according claim 1, further comprising one or more oral care agents chosen from a source of fluoride ions, dental abrasives, flavors, humectants, chelants and combinations thereof.

11. A method of improving the flowability of particulate stannous salts comprising: adding an alumina and a drying aid to a particulate source of a stannous salt.

12. The method according to claim 11, wherein the stannous salt is chosen from stannous chloride, stannous fluoride and/or stannous pyrophosphate.

13. The method according to 12, wherein the stannous chloride is chosen from stannous chloride dihydrate, stannous chloride anhydrous, and combinations thereof.

14. The method according to claim 11, wherein the alumina is chosen from a spray dried, a rotary calcinated, a fumed, a pearled alumina or combinations thereof.

15. The method according to claim 11, wherein the particulate source has an average particle size of from about 0.4 microns (μm) to about 100 μm.

16. The method according to claim 11, wherein the ratio of particulate stannous salt to alumina can be from about 9:1 to 199:1.

17. The composition according to claim 11, wherein the ratio of drying aid to alumina can be from about 10:1 to 2:1.

18. The method according to claim 11, wherein the drying aid is chosen from calcium sulfate, magnesium sulfate, zinc stearate, and combinations thereof.

19. An oral care composition comprising:
a) a particulate source of a stannous salt;
b) an alumina; and
c) a drying aid,
wherein the stannous salt is chosen from stannous chloride, stannous fluoride and/or stannous pyrophosphate, the alumina is chosen from a spray dried, a rotary calcinated, a fumed, a pearled alumina or combination thereof, and the drying aid is chosen from calcium sulfate, magnesium sulfate, zinc stearate, and combinations thereof.

20. The composition according to claim 19, wherein the oral care composition is a toothpaste or gel, a mouth rinse, a mouth spray, a bleaching agent, a lozenge or a chewing gum.
